(19) United States
(12) Patent Application Publication
(10) Pub. No.: US 2006/0275292 A1
(43) Pub. Date: Dec. 7, 2006

(54) Fully human anti-CD3 monoclonal antibodies

(75) Inventor: Terry L. Delovitch, London (CA)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: Amgen Fremont Inc., Fremont, CA (US)

(21) Appl. No.: 11/502,870

(22) Filed: Aug. 10, 2006

Related U.S. Application Data
(63) Continuation of application No. 10/383,247, filed on Mar. 7, 2003, now abandoned.
(60) Provisional application No. 60/362,337, filed on Mar. 8, 2002.

(51) Int. Cl. A61K 39/395; C12P 21/04; C07K 16/28

(52) U.S. Cl. 424/144.1; 530/388.22; 435/70.21

(57) ABSTRACT

Non-toxic anti-CD3 antibody is useful for the treatment, prevention, and reversal of human autoimmune disease. Anti-CD3 antibody is generated by immunizing xenogenic mice capable of developing fully human antibodies. Because the inventive antibodies are not derived from other species, they do not the invoke the immune responses typically associated with humanized or grafted antibodies.

This application claims the priority benefit of U.S. provisional patent application Ser. No. 60/362,337, filed Mar. 8, 2002.

### FIELD OF THE INVENTION

The present invention relates to a novel antibody that is useful in the treatment, prevention, and/or amelioration of autoimmune disease conditions in humans. Such an antibody is generated against the CD3 receptor, which is present on human T cells, and can bind the CD3 receptor specifically. An antibody of the present invention is further characterized by a fully human composition, generated by immunizing a xenogenic, non-human animal with human CD3 receptor protein.

### BACKGROUND OF THE INVENTION

Autoimmune diseases are disorders mediated by self-destructive activities of the immune system. Over the course of an autoimmune disease, the immune system responds to one or more antigen in the human body, recruiting humoral or cellular components of the immune system, with the result of an apoptotic or necrotic destruction of cells. As the immune response progresses, the affected tissue or organ is invaded to an extent that it loses its normal function and renders distinct, detectable symptoms of autoimmunity. The commonly known list of autoimmune diseases includes, but is not limited to, allergenic inflammation, asthma, psoriasis, diabetes mellitus, rheumatoid arthritis, multiple sclerosis, lupus erythmateous, transplant rejection, graft rejection, Gaucher's disease, and glomerulonephritis.

The etiology of various autoimmune diseases has revealed important discoveries regarding the mammalian immune system and its regulation. In essence, it is demonstrated within the art that the immune system exists as a balance between inflammatory (Th1) and protective (Th2) responses therein. These responses are characterized by the association with different immune cells, since Th1 responses elevate cytotoxic T cell activity while Th2 responses promote humoral activity. Furthermore, it is known that specific lymphokines are characteristic of mounting a pro-inflammatory Th1 response (i.e., IL-2, IFN-g, TNF-a) while other such lymphokines mediate the Th2 function that reduces inflammation (i.e., IL-4, IL-5, IL-6, IL-9, IL-10 and IL-13). Furthermore, it is well established that the Th1 and Th2 arms counteract each other, such that suppressing of Th2 lymphokines leads to increased Th1-like cytotoxic activity. Conversely, the protective properties of Th2-like humoral activity also can be increased by suppression of Th1-associated cells and/or lymphokines.

Autoimmune diseases are caused by an abnormal elevation of the Th1 immune response, however, such that Th2 lymphokines are sufficiently suppressed. In various animal models and in human subjects, Th2-associated lymphokines are lowered dramatically in correlation with disease incidence. Thus, exogenous blocking or reversal of the Th1 phenotype restores the immune system's balance and prevents further progression of autoimmunity.

Recent studies have investigated the effect of Th1 modulation on the incidence, progression and onset of autoimmune diseases. Studies investigating the role of CD3 receptor stimulation in the balance between Th1/Th2 and the induction of self tolerance have shown, using various in vivo techniques, that stimulation of the CD3 receptor via anti-CD3 antibodies mediates a decrease in the responsiveness of peripheral T cells and splenocytes against self antigens [Ben-Amor et al., Clinical Experimental Immunology 103:491-8, (1996)]. Treatment of T cells with anti-CD3 antibodies lowered secretion of IL-2 and other pro-inflammatory cytokines in comparison to untreated T cells when stimulated with mitogenic agents. In addition, separate in vivo studies indicate decreased onset of autoimmune disease phenotypes upon treatment of animals with anti-CD3 antibodies. In particular, the progression of autoimmune diabetes within NOD mice is reversed dramatically upon treatment with the anti-CD3 antibodies [Chatenoud et al., Journal of Immunology 158(6):2947-54 (1997)]. The utility of anti-CD3 antibody treatment also has been demonstrated in transplant recipients that are prone to autoimmune rejection of transplant tissue [Chatenoud, Transplant Proceedings. 26(6):3191-3 (1994)].

Thus, anti-CD3 antibody therapy has a demonstrated potential in the context of treating autoimmune disease. The efficacy of anti-CD3 therapy has been limited by in vivo toxicities, however. A well-known anti-CD3 antibody, OKT3, is used routinely in clinical therapy of transplant rejection but is known to mediate dramatic cytokine release in vivo, leading to a “flu-like” syndrome. This effect has been identified with a humoral response against the OKT3 molecule as well as a release of pro-inflammatory cytokines such as TNF-α [Chatenoud, Transplant Proceedings. 25:6-73 (1993); Niaudet et al., Pediatric Nephrology. 7(3):263-267 (1993); Abbs et al. Therapeutic Immunology. 1:325-31 (1994); Herbelin et al., Transplantation. 59(10):1470-75 (1995)]. These physiological toxicities restrict the dosage regimens available to patients with anti-CD3 therapy and limit the overall efficacy of anti-CD3 treatment of autoimmune disease.

There is a current need for a nontoxic anti-CD3 therapy. Because the nature of anti-CD3 therapy-associated toxicity has not been identified precisely, however, the characteristics of such a therapy were not apparent heretofore.

### SUMMARY OF THE INVENTION

Accordingly, it is one objective of the present invention to provide non-toxic anti-CD3 antibodies. In accomplishing these and other objects, there is provided, in accordance with one aspect of the present invention, an anti-CD3 antibody composed entirely of human sequences.

In one aspect of the invention, said anti-CD3 antibody is generated through a process where at least one step involves administration of human CD3 protein, or fragments thereof, to a non-human xenogenic animal.

The inventive anti-CD3 antibody is generated, for example, by immunizing xenogenic mice capable of developing fully human antibodies. More generally, antibody within the invention is developed by immunization of a xenogenic, non-human animal with human CD3 protein, where the animal is capable of generating antibodies with at least one fully human variable region. Subsequently, a high-affinity antibody clone is selected that specifically binds the human CD3 antigen. Methods of treatment, prevention, and reversal of human autoimmune disease also are provided.

In another aspect of the invention, said anti-CD3 antibody is generated using a phage-display approach.

According to one aspect of the invention, a fully human anti-CD3 antibody is administered to human individuals that are either at risk, currently developing or have already developed one or more condition associated with autoimmunity. Such autoimmune diseases include but are not limited to allergenic inflammation, asthma, psoriasis, Type I diabetes, rheumatoid arthritis, multiple sclerosis, lupus erythmateous, transplant or graft rejection, and glomerulonephritis. As is known within the art, such individuals may be identified using identified serological, genetic and biochemical markers indicative of disease susceptibility or progression of one such autoimmune disease condition. It is further an embodiment of this invention that a fully human anti-CD3 antibody is administered into human subjects subsequent to the graft or transplant of foreign tissue, including but not limited to insulin-secreting tissues and/or pancreatic islet cells, in order to prevent the rejection thereof and increase the survival of the graft or transplant in vivo.

### DETAILED DESCRIPTION OF THE INVENTION

This invention provides a non-toxic anti-CD3 antibody useful in the treatment, prevention, and reversal of human autoimmune disease. An anti-CD3 antibody within the invention can be generated by immunizing xenogenic mice capable of developing fully human antibodies. Alternatively, such a non-toxic anti-CD3 antibody may be developed using phase-display methods using antibodies containing only human sequences. Since the inventive antibodies are not derived from non-human species, they do not the invoke the immune responses typically associated with humanized or grafted antibodies when administered to human subjects. A monoclonal anti-human CD3 antibody generated according to the present invention is referred to as “fully human anti-CD3 monoclonal antibody” or “fhCD3mAb.”

The phrase “nucleic acid” denotes DNA and RNA that can either be of single- or double-stranded structure. The terms “protein” and “polypeptide” refer to amino acid polymers, existing in an unfolded or folded spatial organization, with or without catalytic function. The term “antibody” refers to protein molecules derived from a polyclonal or monoclonal population of B cells of mammalian origin. The phrase “antibody fragment” refers to the aforementioned antibody molecules that have been cleaved into different segments, optionally labeled with fluorochrome compounds for the purpose of detection. The term “chemokine” refers to all known chemotactic cytokines expressed within mammalian organisms that mediate the recruitment and infiltration of leukocytes into tissues. The term “chemokine” includes but is not limited to all mammalian members of the C, CC, CXC, and CXXXC families of chemotactic cytokines, classified within the art based upon the distribution of cystine residues therein. The phrase “chemokine receptor” refers to transmembrane proteins, exemplified in the art, that interact with one or more chemokines. The category of “chemokine receptor” includes but is not limited to all chemokine receptors classified within the art as CR, CCR, CXCR and CXXXCR. The term “cytokine” refers to all human cytokines known within the art that bind extracellular receptors upon the cell surface and thereby modulate cell function, including but not limited to IL-2, IFN-G, TNF-a, IL-4, IL-5, IL-6, IL-9, IL-10, and IL-13. The term “cytokine receptor” refers to all human cytokine receptors within the art that bind one or more cytokine(s), as defined herein, including but not limited to receptors of the aforementioned cytokines.

This invention includes methods to produce fhCD3mAb by a process wherein at least one step of the process includes immunizing a transgenic, non-human animal with human CD3, protein. The endogenous heavy and/or kappa light chain loci of this xenogenic non-human animal have been disabled and are incapable of the rearrangement required to generate genes encoding immunoglobulins in response to an antigen. In addition, at least one human heavy chain locus and at least one human light chain locus have been stably transfected into the animal. Thus, in response to an administered antigen, the human loci can rearrange to provide genes encoding human variable regions immunospecific for the antigen. Upon immunization, therefore, the xenomouse produces B-cells that secrete fully human immunoglobulins.

A variety of techniques are well-known in the art for producing xenogenic non-human animals. For example, see U.S. Pat. No. 6,075,181 and No. 6,150,584. By one strategy, the xenogeneic (human) heavy and light chain immunoglobulin genes are introduced into the host germ line (e.g., sperm or oocytes) and, in separate steps, the corresponding host genes are rendered non-functional by inactivation using homologous recombination. Human heavy and light chain immunoglobulin genes are reconstructed in an appropriate eukaryotic or prokaryotic microorganism, and the resulting DNA fragments are introduced into the appropriate host, for example, the pronuclei of fertilized mouse oocytes or embryonic stem cells. Inactivation of the endogenous host immunoglobulin loci is achieved by targeted disruption of the appropriate loci by homologous recombination in the host cells, particularly embryonic stem cells or pronuclei of fertilized mouse oocytes. The targeted disruption can involve introduction of a lesion or deletion in the target locus, or deletion within the target locus accompanied by insertion into the locus, e.g., insertion of a selectable marker. In the case of embryonic stem cells, chimeric animals are generated which are derived in part from the modified embryonic stem cells and are capable of transmitting the genetic modifications through the germ line. The mating of hosts with introduced human immunoglobulin loci to strains with inactivated endogenous loci will yield animals whose antibody production is purely xenogeneic, e.g., human.

In an alternative strategy, at least portions of the human heavy and light chain immunoglobulin loci are used to replace directly the corresponding endogenous immunoglobulin loci by homologous recombination in embryonic stem cells. This results in simultaneous inactivation and replacement of the endogenous immunoglobulin. This is followed by the generation of chimeric animals in which the embryonic stem cell-derived cells can contribute to the germ lines.

In a preferred embodiment of the invention, a B cell clone that expresses human anti-CD3 antibody is removed from the xenogenic non-human animal and immortalized according to various methods known within the art. Such B cells may be derived directly from the blood of the animal or from lymphoid tissues, including but not restricted to spleen, tonsils, lymph nodes, and bone marrow. The resultant, immortalized B cells may be expanded and cultured in vitro to produce large, clinically applicable quantities of fhCD3mAb. Alternatively, genes encoding the immunoglobulins with one or more human variable regions can be recovered and expressed in a differing cell type, including but not restricted to a mammalian cell culture system, in order to obtain the antibodies directly or individual chains thereof, composed of single chain Fᵥ molecules.

In addition, the entire set of fully human anti-CD3 antibodies generated by the xenogenic non-human animal may be screened to identify one such clone with the optimal characteristics. Such characteristics may include binding affinity to the human CD3 protein, stability of the interaction as well as the isotype of the fully human anti-CD3 antibody. Clones from the entire set which have the desired characteristics then can be used as a source of nucleotide sequences encoding the desired variable regions, for further manipulation to generate antibodies with these characteristics, in alternative cell systems, using conventional recombinant or transgenic techniques.

In another aspect of the invention, a fully human anti-CD3 antibody may be generated using a phase-display approach. Such approaches are well-known within the art and outlined under WO92/01047 and U.S. Pat. No. 6,521,404, which are hereby incorporated by reference. In this approach a combinatorial library of phage carrying random pairs of light and heavy chains. The library is then plated and screened using a labeled CD3 peptide.

It is further an object of this invention to generate analogs of fully human anti-CD3 antibodies. The term “analogs” includes fragments, conjugates, isotypes and fusion proteins that are developed using or are derived from the parent fully human anti-CD3 antibody clone or clones outlined above. It is contemplated that Fab fragments of the fhCD3mAb may yield greater therapeutic efficacy than the complete fhCD3mAb containing an Fc region as within the art.

Antibody fragments comprise the antigen-binding portions of an antibody, such as F(ab′)₂, F(ab)₂, Fab′, Fab, and the like. The antibody fragments bind to the same antigen that is recognized by the intact antibody. The term “antibody fragment” also includes any synthetic or genetically engineered protein that acts like an antibody, by binding to a specific antigen to form a complex. For example, antibody fragments include isolated fragments, “Fv” fragments, consisting of the variable regions of the heavy and light chains, recombinant single chain polypeptide molecules in which light and heavy chain variable regions are connected by a peptide linker (“sFv proteins”), and minimal recognition units consisting of the amino acid residues that mimic the hypervariable region.

In another aspect, the invention relates to an immortalized cell line secreting fhCD3mAb where the cell line is an immortalized B cell or is generated by expressing genes encoding such fhCD3mAb within a cell other than a mammalian B cell.

In a preferred embodiment, fhCD3mAb is administered as a pharmaceutical composition. Such pharmaceutical compositions can be formulated using one or more physiologically acceptable carriers or excipients. Preferred compounds are those formulated for parenteral administration by injection, e.g., by bolus injection or continuous infusion. Formulations for injection can be presented in unit dosage form, e.g., in ampules or in multi-dose containers, with an added preservative. The compositions can take such forms as suspensions, solutions or emulsions in oily or aqueous vehicles, and may contain formulatory agents such as suspending, stabilizing or dispersing agents. Alternatively, the active ingredient may be in powder form for constitution with a suitable vehicle, e.g., sterile pyrogen-free water, before use.

The compositions of the present invention also can be prepared as pharmaceutically-acceptable salts. A pharmaceutically-acceptable salt of a composition of the invention means a salt formed between any one or more of the charged groups in the peptide and any one or more pharmaceutically acceptable, non-toxic cations or anions. Organic and inorganic salts include, for example, those prepared from acids such as hydrochloric, sulfuric, sulfonic, tartaric, fumaric, hydrobromic, glycolic, citric, maleic, phosphoric, succinic, acetic, nitric, benzoic, ascorbic, p-toluenesulfonic, benzenesulfonic, naphthalenesulfonic, propionic, carbonic, and the like. Pharmaceutically-acceptable salts may also contain cations including, but not limited to, ammonium, sodium, potassium, calcium, or magnesium.

According to the present invention, moreover, a composition as described can be combined with other chemical or biological entities that are useful in the treatment or prevention of autoimmune disease.

As noted fhCD3mAb of the present invention can be mixed, modified, or introduced into a pharmaceutical composition. Means of delivery for such a composition include but are not limited to microinjection, liposome delivery, subcutaneous injection, intravenous injection, oral administration, inhalation, transdermal application, and rectal administration. A “therapeutically effective” amount may determined by prevention or amelioration of adverse conditions or symptoms of diseases, injuries, or disorders being treated. In keeping with the invention, pharmaceutical compositions and related therapeutic regimens will be optimized, according to conventional practice, as a function of factors such as disease stage and the age, sex and weight of the individual under treatment.

In one embodiment, reagents suitable for the therapies and diagnostics outlined here are packaged into convenient kits providing the necessary materials packaged into suitable containers. Such kits may include suitable supports useful and assisting in performing the therapeutic and diagnostic strategies outlined here.

In another embodiment of the invention, the fhCD3mAb is administered into human subjects to prevent, reduce or decrease the incidence of inflammation therein. It is envisioned herein that fhCD3mAb blocks inflammatory processes in vivo when administered into a human subject before or during the incidence of inflammation. Furthermore, this invention contemplates that administration of fhCD3mAb into human subjects within a localized tissue site shall reduce inflammation-mediated symptoms such as pain, redness and/or swelling within the localized region of the human body.

In another embodiment of the invention, fhCD3mAb is administered into human subjects to prevent, reduce or decrease the activation of cells associated with the human immune system. It is envisioned herein that fhCD3mAb composition contains compounds that interfere with the activation of immune cells when administered in vivo. Accordingly, administration of fhCD3mAb is herein further envisioned as a method of preventing and treating human disease conditions associated with abnormal or deregulated immune cell activation.

In yet another embodiment of the invention, fhCD3mAb is administered to a human individual upon detection of the presence of auto-reactive antibodies within the human individual. Such auto-reactive antibodies are known within the art as antibodies with binding affinity to one or more proteins expressed endogenously within the human individual. In one aspect of the invention, the human individual is tested for the presence of auto-reactive antibodies specifically involved in one or more autoimmune diseases as are well known within the art. In one specific embodiment, a human patient is tested for the presence of antibodies against insulin, glutamic acid decarboxylase and/or the IA-2 protein, and subsequently administered with fhCD3mAb upon positive detection of one or more such auto-reactive antibodies.

In another embodiment of the invention, fhCD3mAb is administered into human subjects to prevent, reduce or decrease the recruitment of immune cells into human tissues. It is envisioned herein that the fhCD3mAb interferes with the recruitment of immune cells when administered in vivo. Therefore, administration of fhCD3mAb is herein further proposed as a method of preventing and treating conditions associated with abnormal or deregulated immune cell recruitment into tissue sites of human disease.

In another embodiment of the invention, fhCD3mAb is administered into human subjects to prevent, reduce or decrease the extravasation and diapedesis of immune cells into human tissues. It is envisioned herein that fhCD3mAb interferes with the extravasation and diapedesis of immune cells when administered in vivo. Therefore, administration of fhCD3mAb is herein further envisioned as a method of preventing and treating conditions associated with abnormal or deregulated immune cell infiltration into tissue sites of human disease.

In another embodiment of the invention, fhCD3mAb is administered into human subjects to prevent, reduce or decrease the effects mediated by the release of cytokines, as defined above, within the human body. It is envisioned that a fhCD3mAb composition of the invention interferes with the in vivo role of mammalian cytokines when the fhCD3mAb is administered in vivo. From this perspective, administering fhCD3mAb is an approach to preventing and treating conditions mediated through abnormal release and production of one or more cytokine(s) in vivo.

In another embodiment of the invention, fhCD3mAb is administered to human subjects to prevent, reduce or decrease the effects mediated by the release of chemokines, as defined above, within the human body. It is envisioned that fhCD3mAb contains compounds that interfere with the in vivo role of mammalian chemokines when the fhCD3mAb is administered in vivo. Therefore, administration of fhCD3mAb is herein further envisioned as a method of preventing and treating conditions mediated through abnormal release and production of one or more chemokine(s) within the human body.

In another embodiment of the invention, fhCD3mAb is administered into human subjects to prevent, reduce or decrease the effects mediated by the release of cytokine receptors, as defined herein, within the human body. It is envisioned herein that fhCD3mAb composition contains compounds that interfere with the in vivo role of mammalian cytokine receptors when the fhCD3mAb is administered in vivo. Therefore, administration of fhCD3mAb is herein further envisioned as a method of preventing and treating conditions mediated through abnormal activation, binding or ligation of one or more cytokine receptor(s) within the human body. It is further envisioned that administration of the fhCD3mAb in vivo will deplete the intracellular signaling mediated by cytokine receptor(s) within such human subject.

In another embodiment of the invention, fhCD3mAb is administered into human subjects to prevent, reduce or decrease the effects mediated by the release of chemokine receptors, as defined herein, within the human body. It is envisioned herein that the fhCD3mAb composition interferes with the in vivo role of mammalian chemokine receptors when the fhCD3mAb is administered in vivo. Therefore, administration of fhCD3mAb is herein further envisioned as a method of preventing and treating conditions mediated through abnormal activation, binding or ligation of one or more chemokine receptor(s) within the human body. It is further envisioned that administration of the fhCD3mAb in vivo shall deplete the intracellular signaling mediated by chemokine receptor(s) within such human subject.

In another embodiment of the invention, fhCD3mAb is administered into a human subject for the purpose of therapeutic intervention of a disease related to the immune system. It is contemplated herein that administration of fhCD3mAb into humans shall yield efficacy in treatment of any disease mediated by dysregulation of the immune system or biological components therein. In one aspect of the invention, fhCD3mAb defined herein is efficacious is treatment of autoimmune diseases including, but not limited to, allergenic inflammation, asthma, psoriasis, Type I diabetes, rheumatoid arthritis, multiple sclerosis, lupus erythmateous, transplant rejection, graft rejection and glomerulonephritis.

In another embodiment, fhCD3mAb is administered to patients that are at risk of developing one of the aforementioned autoimmune diseases. A patient's predisposition to one or more of the aforementioned autoimmune diseases can be determined using genotypic, serological or biochemical markers. For example, the presence of particular HLA subtypes and serological autoantibodies (against insulin, GAD65 and IA-2) are indicative of Type I diabetes.

In another embodiment of the invention, fhCD3mAb is administered to human individuals diagnosed with one or more of the aforementioned autoimmune diseases. Upon diagnosis, fhCD3mAb can be administered to mitigate or reverse the effects of autoimmunity. In one such example, a human individual diagnosed with Type I diabetes is administered with sufficient dose of fhCD3mAb to restore pancreatic function and minimize damage of autoimmune infiltration into the pancreas. In another embodiment, a human individual diagnosed with rheumatoid arthritis is administered with fhCD3mAb to reduce immune cell infiltration into and destruction of limb joints.

In one aspect of the invention, fhCD3mAb is administered to a human individual upon decrease of pancreatic beta-cell function therein. In one embodiment, the individual is tested for beta-cell function, insulin secretion or c-peptide levels as are known within the art. Subsequently, upon notice of sufficient decrease of either the indicator, the human individual is administered with a sufficient dosage regimen of fhCD3mAb to prevent further progression of autoimmune destruction of beta-cell function therein.

In another embodiment of the invention, it is herein disclosed that treatment of a human subject with fhCD3mAb is effective in preventing the rejection of a tissue graft or organ transplant within the human individual. In one instance, it is envisioned that such administration with fhCD3mAb occurs prior to the introduction of a graft or transplant within the patient. In another embodiment, the administration of fhCD3mAb occurs concurrently with graft or tissue introduction to the human individual for an effective period.

It is herein envisioned that the fhCD3mAb composition prevents the rejection of a grant or tissue transplant when administered to the patient prior or subsequent to the introduction of the transplant. In one embodiment, the grafted or transplanted tissue is derived from an organism of the same species as the recipient. It is further embodied that the prevention of transplant rejection is attained through administration of fhCD3mAb when the transplanted or grafted tissue is acquired from a different species of organism than the recipient thereof.

In a further embodiment, the fhCD3mAb composition is administered to a human individual subsequent to tissue transplantation, where the tissue is capable of secreting insulin into the transplant recipient. In one aspect of the invention, it is contemplated that pancreatic islets, or modified derivatives or fractions thereof are transplanted into a patient where fhCD3mAb is concurrently administered or provided subsequent to the transplantation of the tissue.

Other objects, features and advantages of the present invention that become clear as a result of the methods provided herein and depicted in the enclosed drawings are included in this invention. It should be understood that examples and preferred embodiments of the invention herein are given by way of illustration and various alterations and modifications within the spirit of the invention are included as part of the invention herein. Those skilled in the art will recognize alterations and modifications of the invention herein that must however be respected as a part of the present invention.

What is claimed is:

1. An isolated fully human anti-CD3 antibody.

2. A pharmaceutical composition comprising an effective amount of the anti-CD3 antibody of claim 1, wherein said anti-CD3 antibody is generated through a process that comprises administering human CD3 protein or fragments thereof to a non-human, xenogenic animal.

3. A pharmaceutical composition comprising an effective amount of the anti-CD3 antibody of claim 1, wherein said anti-CD3 antibody is generated using phase-display.

4. The anti-CD3 antibody of claim 1, wherein said anti-CD3 antibody includes at least one variable or heavy chain region of human origin.

5. The composition of claim 2, wherein said anti-CD3 antibody is modified through proteolytic cleavage, conjugation or mixing with other reagents.

6. The composition of claim 2, wherein said anti-CD3 antibody is prepared in a therapeutically effective concentration for human therapy.

7. The composition of claim 2, further comprising a pharmaceutically acceptable carrier.

8. The composition of claim 2, wherein said anti-CD3 antibody is administered systemically.

9. The composition of claim 2, wherein anti-CD3 antibody is administered within a specific tissue region of the patient.

10. The composition of claim 2, wherein said anti-CD3 antibody is effective in treating a disease stage that is characterized by inflammation.

11. The composition of claim 10, wherein said disease state results through inflammation of the pancreas or pancreatic tissue.

12. The composition of claim 2, wherein said anti-CD3 antibody is effective in treating a disease stage that is mediated by activation of cells associated with the human immune system.

13. The composition of claim 12, wherein said anti-CD3 antibody is administered systemically.

14. The composition of claim 12, wherein said anti-CD3 antibody is administered within a specific tissue region of the patient.

15. The composition of claim 2, wherein said anti-CD3 antibody is effective in treating a disease stage mediated by the recruitment of immune cells into human tissues.

16. The composition of claim 15, wherein said anti-CD3 antibody is administered systemically.

17. The composition of claim 15, wherein said anti-CD3 antibody is administered within a specific tissue region of the patient.

18. The composition of claim 15, wherein said tissue is the pancreas or pancreatic tissue.

19. The composition of claim 2, wherein said anti-CD3 antibody is effective in treating a diseased stage mediated by the extravasation and diapedesis of immune cells into human tissues.

20. The composition of claim 19, wherein said anti-CD3 antibody is administered systemically throughout the patient.

21. The composition of claim 19, wherein said anti-CD3 antibody is administered within a specific tissue region of the patient.

22. The composition of claim 19, wherein said tissue is the pancreas or pancreatic tissue.

23. The composition of claim 2, wherein said anti-CD3 antibody is effective in reducing a diseased state mediated by release of cytokines within human subjects.

24. The composition of claim 23, wherein said anti-CD3 antibody is administered systemically.

25. The composition of claim 23, wherein said anti-CD3 antibody is administered within a specific tissue region of the patient.

26. The composition of claim 2, wherein said anti-CD3 antibody is effective in treating a diseased state mediated by release of chemokines within human subjects.

27. The composition of claim 26, wherein said anti-CD3 antibody is administered systemically.

28. The composition of claim 26, wherein said anti-CD3 antibody is administered within a specific tissue region of the patient.

29. The composition of claim 2, wherein said anti-CD3 antibody is effective in treating a diseased state mediated by cytokine receptors within human subjects.

30. The composition of claim 29, wherein said anti-CD3 antibody is administered systemically.

31. The composition of claim 29, wherein said anti-CD3 antibody is administered within a specific tissue region of the patient.

32. The composition of claim 2, wherein said anti-CD3 antibody is effective in treating a diseased state mediated by chemokine receptors within human subjects.

33. The composition of claim 32, wherein said anti-CD3 antibody is administered systemically.

34. The composition of claim 32, wherein said anti-CD3 antibody is administered within a specific tissue region of the patient.

35. The composition of claim 2, wherein said anti-CD3 antibody is effective in treating a diseased state or disease mediated by dysregulation of the human immune system.

36. The composition of claim 35, wherein said disease is an autoimmune disease.

37. The composition of claim 36, wherein said autoimmune disease is selected from the group consisting of allergenic inflammation, asthma, psoriasis, Type I diabetes, rheumatoid arthritis, multiple sclerosis, lupus erythmateous, transplant rejection, graft rejection, and glomerulonephritis.

38. The composition of claim 37, where said transplant rejection is of the pancreas or pancreatic tissue.

39. The composition of claim 37, where said graft rejection is of the pancreas or pancreatic tissue.

40. The composition of claim 2, wherein said anti-CD3 antibody is administered to a human individual at risk of developing an autoimmune disease.

41. The composition of claim 40, where said autoimmune disease is selected from the group consisting of allergenic inflammation, asthma, psoriasis, Type I diabetes, rheumatoid arthritis, multiple sclerosis, lupus erythmateous, transplant rejection, graft rejection, and glomerulonephritis.

42. The composition of claim 2, where said anti-CD3 antibody is administered to reverse the onset of an autoimmune disease.

43. The composition of claim 42, where said autoimmune disease is selected from the group consisting of allergenic inflammation, asthma, psoriasis, Type I diabetes, rheumatoid arthritis, multiple sclerosis, lupus erythmateous, transplant rejection, graft rejection, and glomerulonephritis.
